(19) United States
(12) Patent Application Publication
(10) Pub. No.: US 2011/0160833 A1
(43) Pub. Date: Jun. 30, 2011

(54) IMPLANTABLE GRAFT ASSEMBLY

(76) Inventors: Carlos Gonzalez, Richmond (CA); Carlos Vonderwalde, Richmond (CA); Zvi Boms, Beit-hanania (IL)

(21) Appl. No.: 12/668,089

(22) PCT Filed: Jul. 11, 2008
(86) PCT No.: PCT/IB2008/052799

§ 371 Date: Nov. 3, 2010

Related U.S. Application Data
(60) Provisional application No. 60/929,724, filed on Jul. 11, 2007.

(51) Int. Cl. A61F 2/84; A61F 2/82; B23P 11/00

(52) U.S. Cl. 623/1.11; 623/1.15; 29/428

(57) ABSTRACT

An implantable graft-assembly has a) a radially expandable substantially tubular frame (e.g., a stent); and b) a graft having an at least partially curved periphery, such as an oval or circular graft. Also provided are methods of treating aneurysms using such graft assemblies, methods of making the graft assemblies, use of sheets of materials for making the graft assemblies, and methods of mounting graft-assemblies having partial covers such as grafts on delivery devices such as delivery catheters or inside delivery sheaths.

### RELATED PATENT APPLICATION

The present application gains priority from U.S. Provisional Patent Application No. 60/929,724 filed 11 Jul. 2007 which is included by reference as if fully set forth herein.

### FIELD AND BACKGROUND OF THE INVENTION

The present invention relates to the field of intracorporeal implantable medical devices and especially to implantable graft assemblies including a graft having a periphery that is at least partially curved, such as a graft having a circular or elliptical periphery associated with an expandable frame. In some embodiments, the expandable frame is a stent. Some embodiments of graft assemblies of the present invention are useful for deployment in bifurcated vessels or for the treatment of aneurysms, especially cerebral aneurysms.

An aneurysm is a localized ballooning of a blood vessel. Aneurysms can occur in any blood vessel, although they are most common in arteries, particularly in the arteries at the base of the brain (the Circle of Willis) and in the aorta. Approximately 85% of cerebral aneurysms develop in the anterior part of the Circle of Willis and involve the internal carotid arteries and major branches thereof. The most common sites include the anterior communicating artery (30-35%), the bifurcation of the internal carotid and posterior communicating artery (30-35%), the bifurcation of the middle cerebral artery (20%), the bifurcation of the basilar artery, and the remaining posterior circulation arteries (5%).

Once formed, an aneurysm generally continues to grow until the wall of the aneurysm ruptures. Rupture of an aneurysm causes severe pain, internal hemorrhage, and, without prompt treatment, may result in death.

A stent is a substantially tubular radially-expandable device configured for deployment inside the lumen of a bodily vessel or other structure structure. For deployment, a stent is mounted on a deployment catheter, inserted through an incision in the skin and percutaneously guided in an unexpanded state with a small radial dimension through the body to the deployment location. At the deployment location, the stent is expanded to an appropriately-sized expanded state with a larger radial dimension, so as to engage the inner walls of the vessel, acting as a supporting structure to prevent collapse and maintain patency of the vessel lumen, and in some cases to define the lumen.

A first type of stent is the self-expanding stent. When a self-expanding stent is at the deployment location, the stent is released from the catheter and allowed to expand to an expanded state, in a manner analogous to that of a compressed spring. Self-expanding stents have been disclosed, for example, in U.S. Pat. Nos. 4,503,569; 4,580,568; 4,787,899; and 5,104,399.

A second type of stent is expanded from the unexpanded state to an expanded state using an expansion device, typically a catheter-borne balloon. When the stent is at the deployment location, the expansion device is activated inside the bore of the unexpanded stent to exert an outwards radial force to the luminal walls of the stent, causing the stent to expand to an expanded state of a desired radial dimension. Such stents have been disclosed, for example, in U.S. Pat. Nos. 4,655,771; 4,733,665; 4,739,762; 4,800,882; 4,907,336; 4,994,071; 5,019,090; 5,035,706; 5,037,392; and 5,147,385.

Stents are generally of open-walled construction (e.g., slotted or otherwise cut-out tubes, bent wires), allowing material to pass through the openings between the structural elements that define the stent frame.

Covered stents are stent assemblies comprising a stent with a tubular stent cover (also called a jacket) of a synthetic material or of biological tissue, the stent cover covering the openings in the stent body.

One effective method of treatment of aortic aneurysms to prevent rupture or growth thereof is to deploy a covered stent across the neck of the aneurysm. The stent cover seals the aneurysm neck so that the thus-sealed aneurysm does not grow further.

As is known to one skilled in the art, many blood vessels of the body are bifurcated. By “bifurcated” is meant an object that splits to two branches along a length of the object, and generally comprises a trunk vessel from which a branch vessel branches at a bifurcation point. Herein by bifurcated vessel is also meant a ramificated or multiply branched vessel, that is to say a trunk vessel with numerous vessels branching off at various locations along its length where bifurcation refers to a specific branching. An aneurysm may be situated on or near a bifurcation point. A problem with using a covered stent to treat an aneurysm on a bifurcated vessel is that the stent cover may partially or totally obstruct the entrance into the branch vessel, stopping or altering the flow into the branch vessel, increasing pressure at the bifurcation point and causing turbulent flow, factors that may lead to stenosis of the trunk vessel or of the branch vessel or damage to parts of the body dependent on blood from the branch vessel.

Cerebral aneurysms are exceptionally challenging to treat due to the small lumen and exceptional tortuosity of the cerebral vascular system.

It would be desirable to treat cerebral aneurysms by sealing the aneurysm in a single-step procedure involving the use of a covered stent and without occluding side branches branching from the main vessel. The use of covered stents for treatment of aneurysms in the brain has been precluded: the addition of a cover to a stent reduces the stent flexibility and increases the outer diameter (profile) of the stent, see for example “Stent-Graft Placement for Wide-Neck Aneurysm of the Vertebrobasilar Junction” by M. A. Burbelkoa; L. A. Dzyakb; N. A. Zorinb; S. P. Grigorukc; and V. A. Golykb.

In PCT patent application IL2007/000140 of the Inventor are disclosed implantable graft assemblies that are substantially stents having only partial covers. In some embodiments, such implantable graft assemblies comprise a graft (constituting a partial stent cover) secured to a stent, wherein in an expanded state a first portion of the surface area of the frame of the stent is covered by the graft and a second portion of the surface area of the frame is free of the graft. In some embodiments, the covered portion has a circumferential section which is less than the entire circumference of the frame of the stent. In some embodiments the covered portion has a length less than the length of the frame of the stent. Such graft assemblies are exceptionally useful for the treatement of aneuryms of bifurcated and other ramificated and sidebranched vessels. The graft assembly is deployed so that the graft constituting the partial cover seals the neck of the aneurysm without obstructing branch vessels. Additionally, the partial cover gives the graft assembly a relatively low profile and improved flexibility compared to a complete cover, both factors that render some embodiments of such graft assemblies exceptionally useful for treatment of cerebral aneurysms.

Additional stents provided with partial covers have been disclosed in patent publications WO 2007/051179 and US 2003/171801.

Although the teachings of PCT patent application IL2007/000140 of the Inventor provide for highly effective treatment of aneurysms, and especially of cerebral aneurysms, there is always a need for improvement. Specifically, it would be highly advantageous to have a graft assembly such as a covered stent or similar device which is even more suitable for treatment of cerebral aneurysms and/or aneurysms in the proximity of a bifurcation.

### SUMMARY OF THE INVENTION

Some embodiments of the present invention successfully address at least some of the shortcomings of prior art by providing implantable graft assemblies exceptionally useful for the treatement of aneurysms, especially such aneurysms as cerebral aneurysms or aneurysms of bifurcated, ramificated or sidebranched vessels. Some embodiments of the present invention allow for substantial sealing or partially blocking of the neck of an aneurysm on a bifurcated vessel by providing a graft having a relatively small surface area and a shape so as to cause little or no blockage of a branch vessel. Some embodiments of the present invention provide an implantable graft-assembly that has a lower profile and is more flexible due to the small size and shape of the graft, allowing maneuvering through smaller vessels such as found in the brain.

According to some embodiments of the teachings of the present invention there is provided an implantable graft-assembly comprising: a) a radially expandable substantially tubular frame having a distal frame end and a proximal frame end; and b) a graft associated with the frame having an at least partially curved periphery, wherein in an expanded state of the expandable frame a first portion of the surface area of the expandable frame is covered by the graft and a second portion of the surface area of the expandable frame is free of the graft, the first portion having a circumferential section which is less than the entire circumference of the expandable frame.

According to some embodiments of the teachings of the present invention there is also provided a method of treating an aneurysm, comprising: a) providing an implantable graft-assembly comprising: i) a radially expandable substantially tubular frame having a distal frame end and a proximal frame end; and ii) a graft associated with the frame having an at least partially curved periphery wherein in an expanded state of the frame a first portion of the surface area of the frame is covered by the graft and a second portion of the surface area of the frame is free of the graft, the first portion having a circumferential section which is less than the entire circumference of the frame; b) providing a delivery system for deploying the implantable graft-assembly within a blood vessel on which an aneurysm is located; and c) deploying the implantable graft-assembly within the blood vessel using the delivery system, such that the portion of the frame covered by the graft is positioned across a neck of the aneurysm, in some embodiments thereby blocking (at least partially) the neck of the aneurysm from communication with the blood vessel. In some embodiments, the deploying of the implantable graft-assembly is such that a curved portion of the periphery of the graft end faces the direction of flow of blood through the blood vessel. In some embodiments, the deploying of the implantable graft-assembly is such that a curved portion of the periphery of the graft end faces away from the direction of flow of blood through the blood vessel.

According to some embodiments of the teachings of the present invention there is also provided a method of making an implantable graft-assembly; comprising: a) providing a radially expandable substantially tubular frame; b) providing a graft comprising a sheet of material suitable for use as an implantable graft having an at least partially curved periphery; and c) associating (e.g., securing, attaching) the graft with a surface of the frame so that the graft covers a first portion of the surface area of the frame and a second portion of the surface area of the frame is free of the graft, wherein the first portion has a circumferential section which is less than the entire circumference of the frame.

According to some embodiments of the teachings of the present invention there is also provided for the use of a graft comprising a sheet of material having an at least partially curved periphery in the preparation of an implantable graft-assembly, comprising associating (e.g., securing, attaching) the graft with a surface of a radially expandable substantially tubular frame so that the graft covers a first portion of the surface area of the frame and a second portion of the surface area of the frame is free of the graft, wherein the first portion has a circumferential section which is less than the entire circumference of the frame.

In some embodiments of the present invention, the graft is substantially disposed on an outer surface of the frame. In some embodiments of the present invention, the graft is substantially disposed on an inner surface of the frame. In some embodiments of the present invention, portions of the graft are substantially disposed on an outer surface and portions on an inner surface portion of the frame.

In some embodiments of the present invention the graft is substantially a sheet of material. To reduce the profile of the graft-assembly and to increase axial flexibility it is preferred that the graft be relatively thin. In some embodiments, the graft is inherently flat and adopts a curved shape when associated with the expandable frame. In some embodiments, the graft is inherently curved, e.g., has a cylindrical or elliptical cross-section.

In some embodiments, the periphery of the graft is substantially entirely curved. For example, in some embodiments, the graft has a periphery that has a shape selected from the group consisting of circles, ovals, ellipses, oblate ovals, oblate ellipses and oblate circles.

In some embodiments, a sector spanning at least about 90°, at least about 120°, at least about 180° and even at least about 270° of the periphery of the graft is curved, e.g., the periphery of the graft has a shape that is partially curved, for example is substantially that of a polygon (square, rectangle, trapezoid, pentagon, hexagon and so on) having one or more curved edges and/or rounded vertices.

In some embodiments, the graft is associated with the expandable frame so that a curved portion of the periphery of the graft is directed towards the upstream end of the expandable frame. As is discussed below, in some embodiments, the upstream end is the distal end of the expandable frame while in some embodiments, the upstream end is the proximal end of the frame. In some embodiments, the graft is associated with the frame so that a curved portion of the periphery of the graft is directed towards the downstream end of the expandable frame.

In some embodiments of the present invention, the first portion has a length substantially equal to the length of the frame. In some embodiments of the present invention, the first portion has a length no more than about 80%, no more than about 50% and even no more than about 34% of the total length of the frame in an expanded state.

By “unexpanded state” is meant the conformation of the frame when the graft-assembly is associated with a suitable delivery device, such as a delivery balloon catheter (e.g., for balloon-expanded frames such as balloon expanded stents) or inside a delivery sheath (e.g., for self-expanding frames such as self-expanding stents).

By “expanded state” is meant a conformation of the frame when the graft-assembly has been deployed from the delivery device. The diameter of the frame in an expanded state is usually determined by the connection points (e.g., sutures) of the graft to the frame.

In some embodiments, the first portion constitutes no more than about 80%, no more than about 67%, no more than about 50% and even no more than about 34% of the surface area of the frame in an expanded state.

In some embodiments of the present invention, the first portion covers a circumferential section which is less than the entire circumference of the frame in an expanded state. In some embodiments of the present invention, the first portion covers a circumferential section not more than about 330°, not more than about 270°, not more than about 240°, not more than about 180°, not more than about 120° or even covering not more than about 90° of the entire circumference of the frame.

In some embodiments, the frame further comprises a plurality of graft-connecting features distributed over the surface of the frame are and associating the graft to the frame comprises associating (e.g., securing or attaching) the graft to at least one graft-connecting feature in accordance with dimensions of the graft.

In some embodiments of the present invention, the tubular frame is configured to be self-expanding (e.g., analogous to self-expanding stents known in the art). In some embodiments of the present invention, the tubular frame is configured to radially expand by application of an outwards force applied to an inner surface of the tubular frame (e.g., analogous to balloon expandable stents known in the art), for example as would be applied by a standard catheter-mounted balloon. In some embodiments of the method of the present invention, the delivery system comprises an expansion device, for example a balloon catheter.

In some embodiments of the present invention, the tubular frame is substantially a stent.

In some embodiments of the present invention, the graft-assembly includes at least one marker (e.g., functionally associated with the graft, the frame or both) detectable by a medical imaging modality, such as radiation emission, X-ray transmission, magnetic resonance imaging or ultrasound. The marker or markers allow the orientation and position of the graft to be accurately ascertained during deployment. In some embodiments, at least one marker is disposed so as to mark the curved portion of the periphery of the graft directed towards the distal end of the frame. In some embodiments, at least one such marker is disposed so as to delineate the periphery of the graft.

In some embodiments of the present invention, there is an alignment hole penetrating through the graft, preferably positioned substantially near the center of the graft.

In some embodiments of the method of treating an aneurysm of the present invention, the blood vessel is bifurcated, ramificated or sidebranched. Preferably, during deployment the portion of the frame that is free of the graft is positioned at a bifurcation of the bifurcated blood vessel so as not to obstruct with flow (e.g., of blood) between the trunk and branch vessels of the bifurcated vessel. In some embodiments, the aneurysm is a cerebral aneurysm. In some embodiments, the aneurysm is a saccular, fusiform or berry aneurysm.

According to the teachings of the present invention there is also provided for the use of an implantable graft-assembly as described above in the treatment of an aneurysm, especially an aneurysm located on a branched blood vessel, or a cerebral aneurysm, and especially saccular, fusiform or berry aneurysms.

According to some embodiments of the teachings of the present invention there is provided a device for deploying a graft-assembly in a vessel of a mammalian body, comprising:
a) an elongated delivery catheter with a distal end and a proximal end, including: i. a catheter-guiding guide wire lumen; and ii. a graft-assembly deploying mechanism; and
b) a graft-assembly, including: iii. a radially expandable substantially tubular frame having a distal frame end and a proximal frame end; and iv. a graft associated with the frame, wherein a first portion of the surface area of the frame is covered by the graft and a second portion of the surface area of the frame is free of the graft the first portion having a circumferential section which is less than the entire circumference of the frame
   the graft-assembly in an unexpanded state encircling the delivery catheter near the distal end of the delivery catheter and functionally associated with the graft-assembly deploying mechanism; and the elongated catheter configured to control the radial orientation of the graft-assembly inside the body of a mammal when the graft-assembly is functionally associated with the graft-assembly deploying mechanism.

In some embodiment, the graft has an at least partially curved periphery, as described for the graft-assemblies above. In some embodiments, the graft-stent is oriented so that a curved part of the periphery is directed towards the distal end of the catheter. In some embodiments, the graft-stent oriented so that a curved part of the periphery is directed towards the proximal end of the catheter.

In some embodiments, the configuration for radially orienting the graft-assembly comprises a rotation mechanism configured for controllable rotation of the graft-assembly inside a body of a patient.

In some embodiments, the configuration for radially orienting the graft-assembly comprises an orientation guide wire lumen associated with the delivery catheter, including a proximal port and a distal port near the distal end of the delivery catheter.

In some embodiments, the distal port is in-line with the graft (in some embodiments in-line with the longitudinal axis of the graft) allowing a guide wire emerging from the distal port in parallel to the delivery catheter to pass over a portion of the graft.

In some embodiments, the graft comprises an alignment hole penetrating through the graft and the distal port is in-line with the alignment hole, allowing a guide wire emerging from the distal port in parallel to the delivery catheter to pass between the delivery catheter and the proximal end of the graft under the proximal edge of the graft to emerge out through the alignment hole.

In some embodiments, the distal port is in-line with the second portion of the surface area of the frame that is free of the graft, allowing a guide wire emerging from the distal port in parallel to the delivery catheter to pass over an are that is free of the graft, or to pass between the delivery catheter and the proximal end of the frame and to emerge out through gaps in the frame. In some such embodiments, the distal part is at about 90° from a longitudinal axis of the graft. In some such embodiments, the distal part is at about 180° from a longitudinal axis of the graft.

In some embodiments, the tubular frame is self-expanding and the elongated delivery-cather is configured for delivery of self-expanding tubular frames, for example the graft-assembly deploying mechanism comprises a sheath known in the art of self-expanding stents.

In some embodiments, the tubular frame is expanded by application of an outwardly radial force and the elongated delivery-cather is configured for delivery of such tubular frames, for example comprising an expansion-balloon mounted on the catheter as a component of the graft-assembly deploying mechanism.

Unless otherwise defined, all technical and scientific terms used herein have the same meaning as commonly understood by one of ordinary skill in the art to which this invention pertains. In case of conflict, the patent specification, including definitions, will control.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Herein the terms “jacket”, “graft”, and “cover” may, in some instances, be used interchangeably.

### BRIEF DESCRIPTION OF THE FIGURES

Some embodiments of the invention are herein described, by way of example only, with reference to the accompanying figures. The description, together with the figures, makes apparent how embodiments of the invention may be practiced to a person having ordinary skill in the art. It is stressed that the particulars shown in the figures are by way of example and for purposes of illustrative discussion of embodiments of the invention.

In the figures:

FIGS. 1A and 1B depict graft assemblies useful in implementing the teachings of the present invention including an expandable frame that is substantially a stent;

FIG. 1C depicts a graft-assembly useful in implementing the teachings of the present invention including an expandable frame that substantially comprises two terminal expandable rings joined by a graft-support section;

FIG. 1D depicts an expandable frame of graft-assembly useful in implementing the teachings of the present invention including fifteen expandable rings;

FIG. 1E depicts, in side cross section, a graft-assembly useful in implementing the teachings of the present invention including an expandable frame that is substantially a stent, where a graft with an entirely curved periphery in the shape of an oval contacts an outer surface of the frame;

FIG. 1F depicts, in side cross section, a graft-assembly useful in implementing the teachings of the present invention including an expandable frame that is substantially a stent, where a graft with an entirely curved periphery in the shape of an oval contacts an inner surface of the frame;

FIG. 1G depicts, in side cross section a graft-assembly useful in implementing the teachings of the present invention including an expandable frame that is substantially a stent including a graft with an entirely curved periphery in the shape of an oval, where portions of the graft contact an outer surface and portions contact an inner surface of the frame;

FIGS. 2A, 2B, 2C, 2D, 2E, 2F, 2G and 2H depict grafts useful in implementing the teachings of the present invention;

FIG. 3A is a depiction of a ramificated artery with three branch vessels, three bifurcations and with an aneurysm on the trunk vessel in which a graft-assembly of the present invention including a circular graft is deployed;

FIG. 3B is a depiction of a ramificated artery with four branch vessels, four bifurcations and with an aneurysm on the trunk vessel in which a graft-assembly including an elliptical graft is deployed;

FIGS. 4A and 4B depict components of a delivery system allowing in vivo rotation of a graft-assembly attached thereto;

FIG. 5 depicts components of a delivery system having an orientation guide wire that emerges from a distal port of an orientation guide wire lumen proximally to the graft-assembly in-line with the longitudinal axis of the graft of the graft assembly;

FIGS. 6A and 6B depict components of a delivery system having an orientation guide wire that emerges from a distal port of an orientation guide wire lumen in-line with an alignment hole in the graft of the graft-assembly, allowing the orientation guide wire to pass out through the alignment hole;

FIG. 7 depicts components of a delivery system having an orientation guide wire that emerges from a distal port of an orientation guide wire lumen at about 180° from the longitudinal axis of the graft of the graft-assembly;

FIG. 8A depicts components of a delivery system, in cross section, of a self-expanding graft-assembly having an orientation guide wire passing through an alignment hole in the graft and through an orientation guide wire port in the deployment sheath; and

FIG. 8B depicts components of a delivery system, in cross section, of a self-expanding graft-assembly having an orientation guide wire passing through an orientation guide wire port in the deployment sheath located at about 180° from the longitudinal axis of the graft of the graft-assembly.

### DESCRIPTION OF SOME EMBODIMENTS OF THE INVENTION

Aspects of the present invention relate to implantable graft-assemblies and methods of using an implantable graft-assembly, in some embodiments exceptionally useful for deployment in intracranial blood vessels (including bifurcated, ramificated or sidebranched blood vessels) and in bifurcated, ramificated or sidebranched bodily vessels, especially for the treatment of aneurysms.

The principles, uses and implementations of the teachings of the present invention may be better understood with reference to the accompanying description and figures. Upon perusal of the description and figures present herein, one skilled in the art is able to implement the teachings of the present invention without undue effort or experimentation. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

As noted in the introduction above, in PCT patent application IL2007/000140 of the Inventor are taught stent assemblies including a tubular frame (such as a stent) and a graft, where the graft covers only a portion of the surface of the frame. In some embodiments, the graft is shorter than the length of the stent and/or the graft covers a circumferential section of the frame which is less than the entire circumference of the frame. Such graft assemblies have a lower delivery profile and are more flexible, allowing such assemblies to be maneuvered, for example, into the cerebral vasculature and deployed therein, especially so that the graft seals-off an aneurysm neck. The fact that the graft only covers a portion of the frame, provides an added advantage as such a graft-assembly is easily deployed in a bifurcated vessel blocking an aneurysm neck but not significantly obstructing branch vessels.

An aspect of the present invention relates to implantable graft assemblies, exceptionally useful for deployment in cranial blood vessels or in bifurcated bodily vessels, comprising a) a radially expandable substantially tubular frame having a distal frame end and a proximal frame end; and b) a graft that constitutes a partial cover for the frame having an at least partially curved periphery (the curved part of the periphery substantially devoid of discontinuities or angularities) wherein in an expanded state a first portion of the surface area of the frame is covered by the graft and a second portion of the surface area of the frame is free of the graft, the first portion having a circumferential section which is less than the entire circumference of the frame. In some embodiments, the first portion constitutes no more than about 80%, no more than about 67%, no more than about 50% and even no more than about 34% of the surface area of the frame. In some embodiments, the frame is self-expanding, analogous to self-expanding stents. In some embodiments, the frame is configured to radially expand by application of an outwards force applied to an inner surface of the frame, such as, for example, by a balloon catheter.

In some embodiments, the graft is associated with the expandable frame so that a curved portion of the periphery of the graft is directed towards the upstream end of the expandable frame. Depending on the embodiment and where the assembly is to be deployed, the upstream end is the distal end of the expandable frame while in some embodiments, the upstream end is the proximal end of the frame.

For example, in embodiments where a graft-assembly enters the body of a patient through a femoral artery and is deployed in an iliac artery or the aorta, the distal end of the frame is the upstream end of the frame as blood flows from the heart, down the aorta towards the feet.

For example, in embodiments where a graft-assembly enters the body of a patient through a femoral artery, passes the aorta and enters a carotid artery, for example for deployment in the brain, the proximal end of the frame is the upstream end of the frame as blood flows from the heart, through the aorta and up through the carotid artery into the brain.

In some embodiments, the graft is associated with the frame so that a curved portion of the periphery of the graft is directed towards the downstream end of the expandable frame.

An aspect of the present invention relates to a method of treatment of an aneurysm substantially comprising deploying a graft-assembly as described above using a delivery system (such as a catheter known in the art for deploying stents) such that the portion of the frame covered by the graft is positioned across a neck of the aneurysm thereby blocking (at least partially) the neck of the aneurysm from communication with the blood vessel.

In some embodiments, the deploying of the implantable graft-assembly is such that a curved portion of the periphery of the graft end faces the direction of flow of blood through the blood vessel. In some embodiments, the deploying of the implantable graft-assembly is such that a curved portion of the periphery of the graft end faces away from the direction of flow of blood through the blood vessel.

The fact that only a fraction of the surface area of a frame of a graft-assembly is covered by a graft leads to a more flexible graft-assembly having a relatively small profile allowing increased maneuverability for delivery and deployment. Further, the relatively small surface area of a graft allows a graft-assembly to be deployed near a bifurcation of a blood vessel without fear of obstructing a branch vessel. It is important to note that a curved periphery graft of a graft-assembly is smaller and the respective graft-assembly is more flexible when compared to a graft-assembly of PCT patent application IL2007/000140 of the Inventor configured to block a similar-sized aneurysm neck.

Unexpectedly, some embodiments of a graft-assembly of the present invention may have a reduced chance (when compared to graft-assemblies comprising grafts not having a curved periphery) of generating thromboses. Such an effect, when present, might be related to the curved periphery of the graft. Prior art grafts used as partial stent covers have straight edges and discontinuous vertices. Although not wishing to be held to any one theory, under certain conditions the pointed vertices of the prior art grafts may interfere with the flow of blood leading to turbulent flow that increases the risk of generating thromboses. Also, under certain condition, the flow of blood past discontinuities of a prior art graft may lead to turbulent flow that increases the risk of generating thromboses. Also, under certain conditions, the flow of blood past the curved periphery of a graft of a graft-assembly, especially when a curved portion of the periphery faces upstream towards the flow of blood, may be less turbulent and consequently have a reduced chance of generating thromboses.

In some embodiments, the periphery of a graft of a graft-assembly is only partially curved, in some embodiments only a sector spanning at least about 90°, at least about 120°, at least about 180° and even at least about 270° of the periphery of the graft. Typical such grafts have shapes of a polygons (e.g., square, rectangle, trapezoid, pentagon, hexagon and so on) having one or more rounded edges and/or rounded vertices. In some such embodiments, a portion of the graft that is near the upstream end of the tubular frame (the end destined to face the flow of blood) is curved, so as to reduce the chance of thrombus generation otherwise potentially caused by turbulent flow past discontinuites of the graft periphery.

Preferably, the periphery of a graft of a graft-assembly is substantially entirely curved. For example, in some embodiments, the graft has a periphery that has a shape selected from the group consisting of circles, ovals, ellipses, oblate ovals, oblate ellipses and oblate circles.

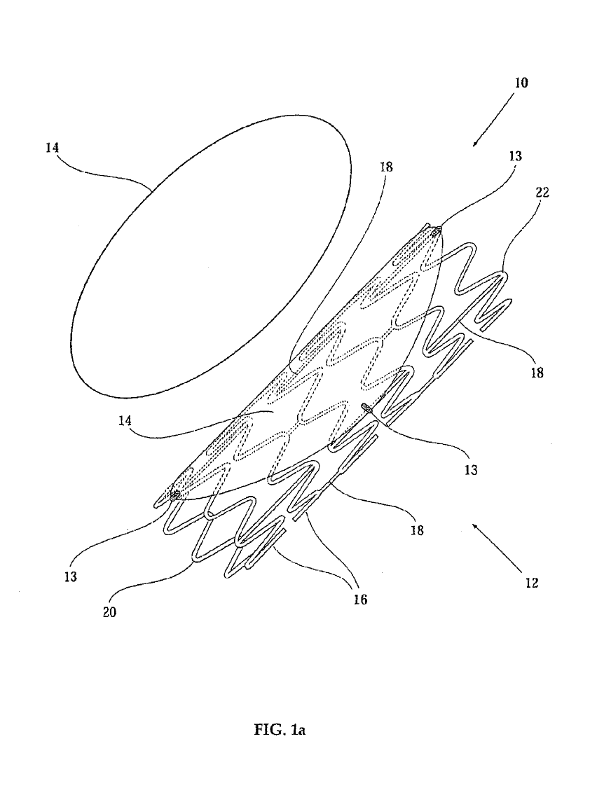

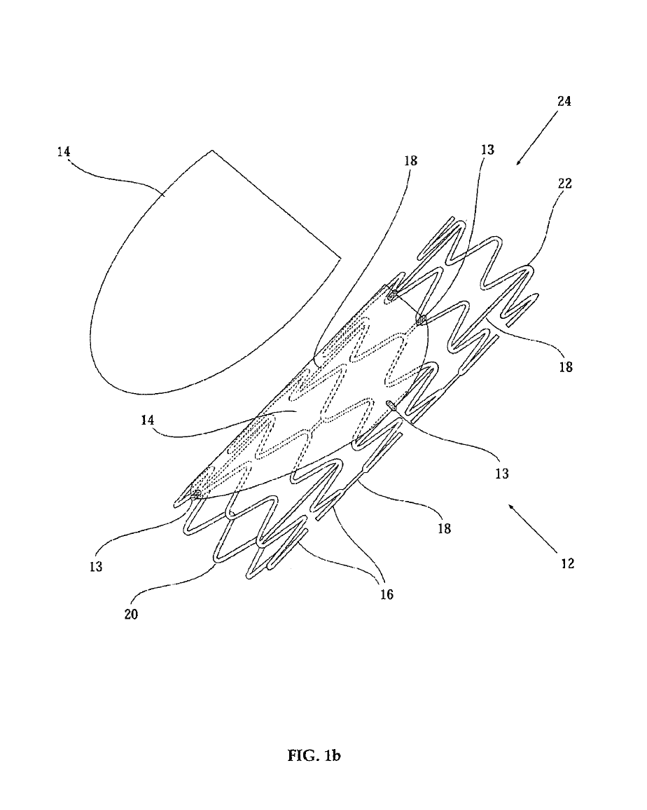

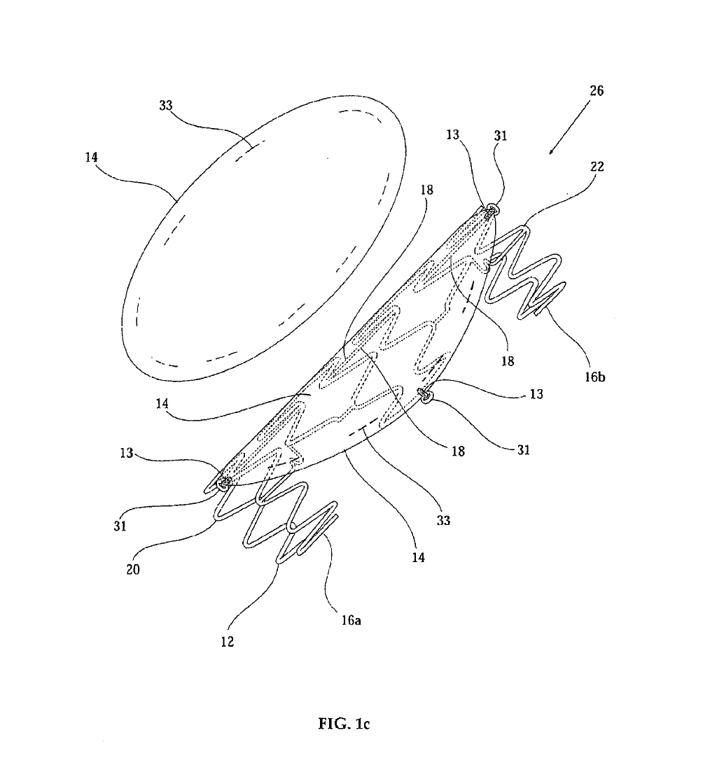

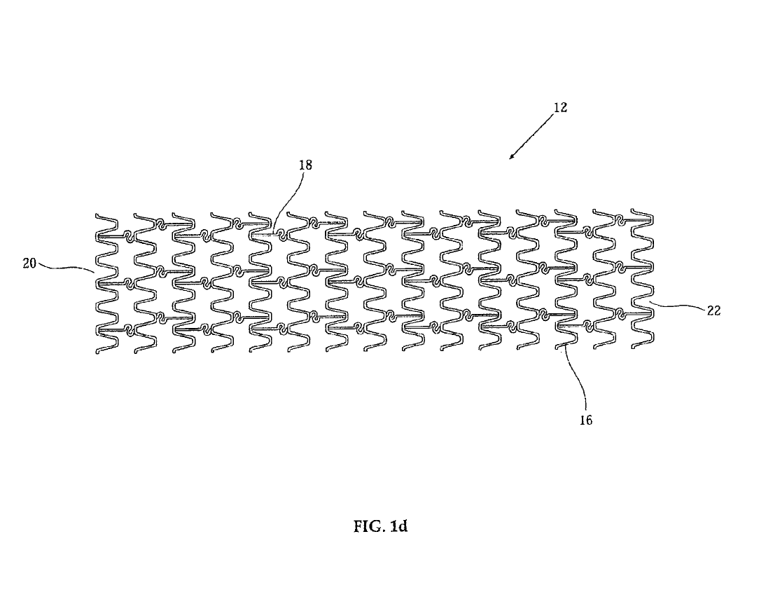

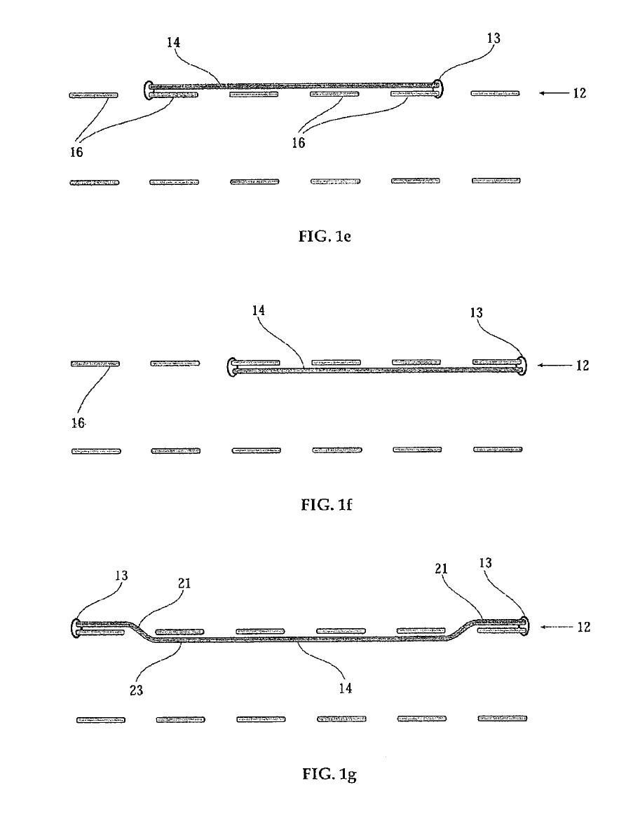

In FIG. 1A, an embodiment of an implantable graft-assembly 10, is depicted, comprising a frame 12 and a graft 14, where graft 14 is shown both alone and associated with (secured with the help of sutures 13) to frame 12.

Frame 12, laser cut from a single tube of stainless steel having 0.1 mm thick walls, is similar to stents known in the art where six individual expandable rings 16 together with struts 18 constitute a radially expandable substantially tubular frame having a proximal frame end 20 and a distal frame end 22. Each expandable ring 16 is made up of a sinusoidally undulated elongated element. Rings 16 are mutually associated with longitudinal struts 18 arrayed so that the length of frame 12 stays substantially the same when radially expanded. In a typical embodiment, frame 12 is 12 mm long (in an axial distance from distal frame end 20 to proximal frame end 22) and has a 6.7 mm diameter in an expanded state. Frame 12 of graft-assembly 10 is configured to be expanded by application of an outwardly radial force, for example as applied by a balloon catheter known in the art of stenting.

Graft 14 is a substantially flat oval patch of 0.1 mm thick crosslinked pericardium (thinned in accordance with the teachings of U.S. Pat. Nos. 6,468,300 and 6,254,627 of the Inventor). Graft 14 is substantially as long as frame 12. As seen in FIG. 1, the entire periphery of graft 14 is curved so as to describe an ellipse. The width of graft 14 is approximately 50% the circumference of frame 12 in an expanded state. As a result, when associated with frame 12 as depicted in FIG. 1A, a first portion of the surface area of frame 12 is covered by graft 14, the first portion having a circumferential section which is less than the circumference of frame 12, and the first portion constitutes approximately 34% of the surface area of frame 12 in the expanded state.

Graft 14 is secured to frame 12 with the help of sutures 13 that pass through graft 14 and around maxima of rings 16. In such a way, maxima of rings 16 function as graft-connecting features.

In FIG. 1B is depicted an embodiment of a graft-assembly, graft-assembly 24. In contrast to graft-assembly 10 depicted in FIG. 1A, in graft-assembly 24, graft 14 is a truncated ellipse about 80% the length of frame 12 so that the periphery of graft 14 is only partially curved in a sector describing approximately 270° of the periphery of graft 14. In FIG. 1B, it is seen that a curved part of graft 14 faces proximal frame end 20 while the non-curved part is near distal frame end 22. For deployment (for example, in the brain), graft-assembly 24 is mounted on a delivery device (such as a delivery catheter) so that distal frame end 22 is near the distal end of the delivery device. Graft-assembly 24 is maneuvered through an incision in a femoral artery of a patient, upstream through the aorta and into a carotid artery. Once in the carotid artery, graft-assembly 24 is maneuvered downstream to a deployment location. Once deployed, distal frame end 22 and the non-curved part of graft 14 are downstream (facing away from the flow of blood), while proximal frame end 20 and a curved part of graft 24 are upstream (facing towards the flow of blood).

In FIG. 1C is depicted an embodiment of a graft-assembly 26 having an exceptionally low-profile and flexible (and thus maneuverable) frame 12. Graft 14 of graft-assembly 26 is substantially the same as graft 14 of graft-assembly 10 depicted in FIG. 1A. However, in contrast to frame 12 of graft-assembly 10 depicted in FIG. 1A, frame 12 of graft-assembly 26 comprises only two terminal radially expandable rings 16: proximal ring 16a defining proximal frame end 20 and distal ring 16b defining distal frame end 22 where rings 16a and 16b are associated through struts 18 and graft support frame 28 (depicted underneath graft 14) made up of two partial-sector rings, struts 18 and eyelets 31 that may be used as graft-connecting features. Frame 12 is a self-expanding frame, made in a manner and from materials similar to those known in the art of self-expanding stents. Near the periphery of graft 14 are a plurality of radio-opaque staples (e.g., of platinum, platinum-iridium, tungsten wire) that function as markers 33 that are detectable by ultrasound and X-ray transmission medical imaging modalities. Markers 33 allow the orientation and position of graft 14 to be accurately ascertained during deployment.

In FIG. 1D is depicted an embodiment of a frame 12 suitable for use as a frame of a graft-assembly of the invention. Frame 12, laser cut from a single tube of stainless steel having 0.1 mm thick walls, is similar to stents known in the art such as described in U.S. Pat. No. 6,375,677 and as sold under the name BiodivYsio™ AS (evYsio Medical Devices ULC, Vancouver, Canada), where fifteen individual expandable rings 16 together with struts 18 constitute a radially expandable substantially tubular frame having a proximal frame end 20 and a distal frame end 22. Each ring 16 is made up of a sinusoidally undulated elongated element. Rings 16 are mutually associated with longitudinal struts 18. A typical embodiment of a frame 12 depicted in FIG. 1D is about 27 mm long, has an expanded diameter of about 6.6 mm, has struts 18 of a width of 0.076 mm while the width of the sinusoidally undulated elongated element making up rings 16 is 0.114 mm and the length of the portion of the element perpendicular to axis of frame 12 is 0.165 mm. Frame 12 depicted in FIG. 1D is configured to be expanded by application of an outwardly radial force, for example as applied by a balloon catheter known in the art of stenting.

In FIGS. 1A, 1B and 1C a first portion of the surface area of a frame 12 is covered by a graft 14, the first portion having a circumferential section which is less than the circumference of frame 12, and the first portion constitutes approximately 34% of the surface area of frame 12 in the expanded state. In some embodiments, the first portion constitutes no more than about 80% no more than about 67%, no more than about 50% and even no more than about 34% of the surface area of the frame in the expanded state.

In FIGS. 1A and 1C graft 14 is approximately as long as frame 12. In FIG. 1B graft 14 is approximately 80% of the length of frame 12. In some embodiments, a graft 14 has length of no more than about 75%, no more than about 50% and even no more than about 34% of the length of a respective frame 12 in an expanded state.

In FIGS. 1A, 1B and 1C the width of graft 14 is approximately 50% of the circumference of frame 12 in an expanded state so as to cover an approximately 180° circumferential section of frame 12. In some embodiments, a graft 14 has a width so as to cover a circumferential section of no more than about 330°, not more than about 270°, not more than about 240°, not more than about 180°, not more than about 120° or even covering not more than about 90° of the circumference of the frame in an expanded state.

In FIGS. 1A, 1B and 1C, grafts 14 are disposed on and contact an outside surface of a respective frame 12. In FIG. 1D, an additional graft-assembly is depicted in side cross section, where graft 14 is disposed on and contacts an outside surface of frame 12 defined by rings 16. In FIG. 1E, an additional graft-assembly is depicted in side cross section, where graft 14 is disposed on and contacts an inner surface of frame 12 defined by rings 16. In FIG. 1F, an additional graft-assembly is depicted in side cross section, where portions 21 of graft 14 are disposed on and contacts an outer surface of frame 12 defined by rings 16 while portion 23 of graft 14 is disposed on and contact an inner surface of frame 12, in a fashion similar to the teachings of U.S. Pat. No. 6,699,277 of the Inventor and as implemented in the commercially available Over and Under™ stent (Design & Performance (Cyprus) Ltd).

In FIGS. 1A, 1B, 1C, 1D, 1E and 1F grafts 14 are secured to frames 12 with the help of sutures 13 as graft-securing components. In some embodiments, instead of sutures 13, a graft 14 is secured to a respective frame 12 with the help of other suitable graft-securing components such as hooks, piercing members, clamps, adhesives, staples, tacks, pins and bending members, or other applicable mechanical mean or combinations thereof. In some embodiments, the graft-securing components are components distinct from the tubular frame, such as sutures 13. In some embodiments, the graft-securing components are components attached to the frame, for example piercing members attached to the frame by welding. In some embodiments, the graft-securing components are integrally formed to the frame, for example piercing members integrally formed with the frame by a laser cutting process.

In some embodiments, the tubular frame is provided with features that together with graft-securing components assist in securing a graft to a tubular frame.

In some embodiments, such features are related to the shape of the frame, e.g., the frame has one or more components that are sinusoidal, zigzag and the like (for example, as disclosed in PCT/IB012/00315 published as WO 01/66037 of the Inventor) and the features are the maxima of the shape. For example, in FIGS. 1A, 1B and 1C such features are maxima of rings 16.

In some embodiments, such features are specific dedicated components (in some embodiments integrally formed, in some embodiments separate components secured to the frame) such as as open eyelets, closed eyelets, open loops, closed loops and the like. For example, in FIG. 1C such features are eyelets 31 integrally formed with frame 12.

In the embodiments depicted in FIGS. 1A, 1B and 1C, frames 12 comprise rings 16 fashioned from a single laser-cut tube of a suitable material such as Nitinol, stainless steel or a cobalt-chromium alloy. In some embodiments, a frame 12 of a graft-assembly is fashioned in another way, for example fashioned from wires of a suitable material bent into a desired shape.

Generally, any suitable expandable frame may be used in implementing the teachings of the present invention. For example, in FIG. 1C, frame 12 comprises two radially expandable rings 16 each defined by a sinusoidally undulated elongated element, the entire frame configured to be self-expanding. For example, FIGS. 1A and 1B, frames 12 comprises six radially expandable rings 16 each defined by a sinusoidally undulated elongated element constituting a stent, all configured to be radially expand upon application of an outwards force to the luminal surface of rings 16.

In some embodiments (e.g., device 26 of FIG. 1C) the tubular frame is configured to be self-expanding (e.g., analogous to self-expanding stents known in the art). In some embodiments (such as in FIGS. 1A and 1B), the tubular frame is configured to radially expand by application of an outwards force applied to an inner surface of the tubular frame (e.g., analogous to balloon expandable stents known in the art), for example as applied by a standard catheter-mounted balloon

Two important parameters used when selecting or designing an expandable frame for use as the frame of a graft-assembly are the expanded and unexpanded diameters of the frame.

Generally it is important that the unexpanded diameter of a frame be as small as possible to ease navigation through the bodily lumen to the deployment location yet the unexpanded diameter must be large enough to allow threading of the frame onto a delivery catheter and, if necessary, a frame-expanding device such as a stent-expanding balloon.

Generally, any given frame has a wide range of expanded diameters when not associated with a graft. That said, the approximate expanded diameter of a graft-assembly including a graft is generally determined by the width of the graft and by the locations where the graft is connected to the frame. When used, the expanded diameter of a frame subsequent to deployment is determined by the user of the graft-assembly according to medical criteria including the natural size of the lumen of the vessel in which the graft-assembly is deployed. Thus, when deployed the graft-assembly may be expanded to slightly less than the expanded diameter in which case the graft may not be taut or slightly greater than the expanded diameter, in which case the graft may be somewhat stretched.

In some embodiments for deployment in the intracranial vasculature, the tubular frame of a graft-assembly generally has an unexpanded diameter (that is to say, the diameter on a delivery device, for example crimped onto a balloon of delivery catheter or inside a delivery sheath for self-expanding stents) that is no greater than about 2 mm and even no greater than about 1 mm. Suitable such tubular frames generally have a maximal expanded diameter of approximately twice to six times, or even more the unexpanded diameter.

For use in vessels other than those of the brain, generally any type of stent known in the art is useful as a frame of a graft-assembly of the present invention. Such stents include but are not limited to stents marketed by affiliates (e.g., Cordis) of Johnson & Johnson, Guidant (Indianapolis, Ind., USA, now an affiliate of Boston Scientific Corp. and Abbott Laboratories), Medtronic (Minneapolis, Minn., USA), Medinol (Tel Aviv, Israel), Cook Inc. (Bloomington, Ind., USA) and ITGI Medical Design & Performance (Cyprus) Ltd. (Cyprus).

For deployment within intracranial blood vessels thin and flexible frames are preferred such as Neuroform stent (Boston Scientific Corp. Natick, Mass., USA), Neurolink stent (Guidant, Indianapolis, Ind., USA, now an affiliate of Boston Scientific Corp. and Abbott Laboratories) or Boa stent (Balt, Montmorency, France). Particularly preferred is the frame used with the Over and Under™ stent (Design & Performance (Cyprus) Ltd.) which is a low-pressure balloon-expandable stent constructed from an electro-polished stainless steel laser cut tube.

Generally, a graft 14 of a graft-assembly of the present invention is substantially a sheet of material that is suitable for deployment in a body as an implantable graft. In some embodiments, the graft is inherently flat and adopts a curved shape when associated with the expandable frame. In some embodiments, the graft is inherently curved, e.g., has a cylindrical or elliptical cross-section. As discussed above, in some embodiments, the length of a graft 14 is substantially equal to, or shorter than, that of a frame 12.

In some embodiments, a graft 14 is of a stretchable material. In some embodiments, a graft 14 is of a collapsible material, allowing folding of the graft for deployment.

In some embodiments, a graft 14 is of a material allowing proliferation of cells therethrough. That said, it is generally preferred that a graft 14 is of a material is substantially impermeable to fluids so as to effectively close the neck of aneurysm. In some embodiments, a graft 14 constitutes a lining prosthesis that ultimately repairs the blood vessel in which deployed. In some embodiments, a graft 14 is of a material substantially impervious to cell proliferation therethrough. In some embodiments, a graft 14 is of a material substantially impermeable to fluids.

In some embodiments of the invention useful for treating aneurysms, it is preferred that a graft 14 be impervious to cell growth therethrough (to prevent build up and the migration of smooth muscle cells) and impermeable to fluids to effectively seal the aneurysm.

To reduce the profile and to increase axial flexibility of a graft-assembly it is preferred that a graft 14 be as thin as possible. Generally a graft used in implementing the present invention is less than 1 mm thick and even less than 0.45 mm thick, so long as the strength and other mechanical properties are remain sufficient. In some embodiments, the thickness of a graft 14 is up to about 0.45 mm, up to about 0.2 mm and even up to about 0.1 mm.

In some embodiments, a graft 14 is fashioned from a synthetic or polymeric material. Suitable such materials include, but are not limited to, polyfluorohydrocarbon polymers (e.g., polytetrafluorethylene), polyurethanes, elastomers, polyamides (e.g., Nylon), polyesters (e.g., Dacron) and silicone.

In some embodiments, a graft 14 is fashioned from a biological tissue including but not limited to autologous tissue or heterologous tissue such as venous tissue, arterial tissue, serous tissues, serous membranes, pleura, peritoneum, pericardium, dura mater and aortic leaflet. Generally suitable tissue types include but are not limited to equine, porcine, bovine or human tissue. In order to increase the toughness of the tissue, it is often advantageous to treat the tissue, for example with a glutaraldehyde or a phosphate solution, in order to cross-link collagen in the tissue. To reduce the bulk of the graft-assembly, it is often preferred that the tissue be thinned, that is after harvesting one or more layers of the harvested tissue are removed, e.g. by scraping, shaving, slicing or skiving (see U.S. Pat. Nos. 6,468,300 and 6,254,627 of the Inventor).

One type of tissue suitable for implementing the teachings is serous tissue, including serous membranes, pericardium, pleura, peritoneum, dura mater, especially porcine, bovine, equine and human serous tissue.

Serous membranes are made of two strata. The serous stratum of a serous membrane is a very smooth single layer of flattened, nucleated mesothelial cells united at their edges by cement. The serous stratum rests on a tough, fibrous basement layer.

For some embodiments, natural serous membrane comprising both the serous stratum and the basement layer is strong, elastic and thin enough to be useful in fashioning a graft 14.

In some embodiments, a preferred material from which to fashion a graft 14 is serous membranes where at least a portion, and in some embodiments all of the basement layer, has been removed (and is therefore thinned), for example by methods including peeling, shaving as taught in U.S. Pat. Nos. 6,254,627 and 6,468,300 of the Inventor. In embodiments where all the basement layer is removed, a graft 14 is thinned serous membrane that is substantially the serous stratum of serous tissue devoid of a basement layer. Not only is thinned serous membrane sufficiently strong, elastic and even thinner than serous membrane, thinned serous membrane also provides little resistance to radial expansion, making thinned serous membrane exceptionally suitable for use in covering or jacketing self-expanding stents. In some embodiments (especially for deployment within cranial vessels), graft 14 comprises serous tissue, especially serous membrane, devoid of at least a portion of associated basement tissue, and even devoid of all the associated basement tissue to substantially comprise only a serous stratum.

Serous tissue, including thinned serous tissue, resists suture line bleeding, requires no pre-clotting, supports endothelialization and has an excellent host-tissue response. Serous tissue, depending on the type, the source and whether thinned or not, is available in thicknesses of less than 1 mm, less than 0.45 mm, less than 0.2 mm and even less than about 0.1 mm. In some such embodiments, a graft 14 has a thickness of between about 0.05 mm and about 0.20 mm.

In FIG. 1C, graft 14 is provided with a plurality of radio-opaque markers, staples 33 detectable by medical imaging modalities such as ultrasound or X-tray transmission modalities delineating the periphery of graft 14. In some embodiments, a graft-assembly is provided with other markers (e.g., functionally associated with a graft 14, a frame 12 or both) that allow the orientation and position of graft 14 to be ascertained during the deployment process. In some embodiments, at least one marker 33 is disposed in proximity of the distal (curved) end of the assembly. In some embodiments, at least one marker is disposed in proximity of the upstream end of the assembly.

In some embodiments, a graft 14 comprises an alignment hole penetrating through graft 14, which is preferably positioned in the center of the graft. Preferably, the alignment hole has a diameter of no more than about 1 mm, no more than about 0.5 mm, no more than about 0.376 mm. In some embodiments, the alignment hole is about 0.35 mm. The alignment hole is optionally reinforced by a grommet, made, for example, of a material such as biological tissue, muscle tissue, polymer, silicon rubber, metal, gold and titanium. As will described below, in some embodiments of the method of the present invention, an alignment hole is useful in directing the graft to the proper location to block the neck of the aneurysm by allowing a guide wire to pass through the graft into the aneurysm.

In some embodiments, a graft-assembly is configured to release an active agent when deployed. In such embodiments, one or more suitable active agents are releasably contained within the graft and/or the frame. Typical active agents include, for example, anti-thrombogenic agents, anti-angiogenic agents, anti inflammatory agents, anti-coagulant agents and other active agents.

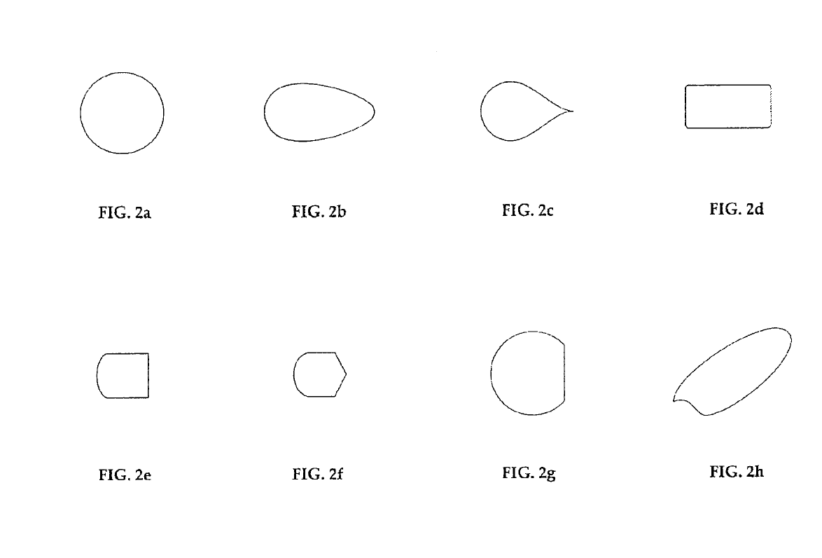

Grafts of many different shapes may be useful in implementing the teachings of the present invention. In FIGS. 1A and 1C, grafts 14 are ellipses. In FIG. 1B graft 14 is a truncated ellipse. Additional graft shapes useful in implementing the teachings of the present invention are depicted in FIG. 2. Embodiments of grafts where the periphery is substantially entirely curved are depicted in FIG. 2A (a circle), FIG. 2B (an oblate oval), FIG. 2C (a tear-drop shape) and FIG. 2H (an ellipse that is curved, that is has an inherently curved cross section and is not flat). Embodiments of grafts where the periphery is only partially curved are depicted in FIG. 2D (a rectangle having rounded vertices), FIG. 2E (a periphery related to a square where the curved distal end is a 90° sector of the periphery of the graft), FIG. 2F (a periphery related to a hexagon where the curved distal end is a 120° sector of the periphery of the graft) and FIG. 2G (a periphery related to a truncated circle where the curved distal end is a 270° sector of the periphery of the graft).

Methods of making a graft-assembly of the present invention are clear to one skilled in the art upon perusal of the description herein. Generally, a graft having an at least partially curved periphery is used in the preparation of an implantable graft-assembly, by associating the graft to a radially expandable substantially tubular frame so that the graft contacts a first portion of the surface area of the frame, wherein the first portion has a circumferential section which is less than the entire circumference of the frame.

A method of making a graft-assembly of the present invention generally comprises a) providing a radially expandable substantially tubular frame (as described above); providing a graft comprising a sheet of material suitable for use as an implantable graft having an at least partially curved periphery (as described above); and c) associating (e.g., securing, attaching) the graft with a surface of the frame so that the graft covers a first portion of the surface area of the frame and a second portion of the surface area of the frame is free of the graft, wherein the first portion has a circumferential section which is less than the entire circumference of the frame in an expanded state. In some embodiments, the graft is associated with the frame so that a curved portion of the periphery of the graft is directed towards a distal end of the frame. In some embodiments, the graft contacts the tubular frame primarily from the luminal side (as an internal jacket). In some embodiments, the graft contacts the tubular frame primarily from the outer side (as an external jacket). In some embodiments, the graft contacts from both the luminal side and the outer side, analogously to the described in U.S. Pat. No. 6,699,277 of the Inventor.

As a graft of a graft-assembly of the present invention covers only a portion of the circumference of the frame, associating the graft to the frame comprises associating the edges to the frame in such a way that between the edges of the graft is a gap, generally through which is apparent a portion of the frame so that when the graft-assembly is deployed the graft covers only a portion of the circumference of the frame.

Generally, a graft of a graft-assembly is secured to a respective frame with graft-securing components such as sutures, hooks, piercing members, clamps, adhesives, staples, tacks, pins and bending members, or other applicable mechanical mean or combinations thereof. In some embodiments, the graft-securing components are components distinct from the tubular frame, such as sutures. In some embodiments, the graft-securing components are components attached to the frame as taught in U.S. Pat. No. 6,929,658 of the Inventor for example piercing members attached to the frame by welding or graft-securing components integrally formed with the frame, for example piercing members integrally formed with the frame by a laser cutting process. In some embodiments, the graft-securing components are clamps, for example as described in the PCT Patent Application IL2007/000140 of the Inventor.

In some embodiments, the tubular frame of a graft-assembly is provided with features (e.g., eyelets, loops, the shape of components of the frame) that together with graft-securing components assist in securing a graft to the tubular frame, as discussed above.

Some embodiments of a graft-assembly are configured to release an active agent when deployed. In some such embodiments, one or more suitable active agents are releasably associated with the graft and/or the frame. Suitable active agents may by associated with a graft-assembly during the manufacturing process, for example as a coating or by impregnating one of the components with an active agent or immediately before deployment of the graft-assembly. For example, in some embodiments, the graft-assembly is immersed for a period of time in an active agent containing solution so as to absorb or adsorb the active agent into the graft and/or frame.

Some embodiments of the implantable graft-assembly optionally comprise a coating on the frame and/or graft. Suitable coatings include, for example, anti-thrombogenic coatings, anti-angiogenic coatings, anti inflammatory coatings, anti-coagulant coatings and other active agent delivering coatings.

Some embodiments of implantable graft assemblies are useful for the treatment of aneurysms, particularly aneurysms which are situated on a bifurcated blood vessel, and especially aneurysms of the cerebrovascular system. Such aneurysms are not amenable to the use of standard covered stents, since such would potentially block the flow of blood into branches leading off the stented vessel, resulting in severe clinical consequences. When deployed within a blood vessel on which an aneurysm is situated, some embodiments of the implantable graft-assembly seals off or at least partially blocks the neck of the aneurysm, thereby preventing rupture or growth of an unruptured aneurysm, or further bleeding of a ruptured aneurysm. For treatment of an aneurysm located on a bifurcated blood vessel, the implantable graft may be positioned so as to seal or cover, at least partially, the neck of the aneurysm, without blocking blood vessels branching off the stented vessel.

Thus, according to the teachings of the present invention there is also provided for the use of an implantable graft-assembly as described above in the treatment of an aneurysm.

Generally, the method of treating an aneurysm of the present invention, comprises: a) providing an implantable graft-assembly as described above; b) providing a delivery system (generally comprising a delivery catheter) for deploying the implantable graft-assembly within a blood vessel on which an aneurysm is located; and c) deploying the implantable graft-assembly within the blood vessel using the delivery system, such that the portion of the frame covered by the graft is positioned across a neck of the aneurysm. In some embodiments where the blood vessel is bifurcated, the portion of the frame that is free of the graft is preferably positioned at a bifurcation of the bifurcated blood vessel so as not to obstruct flow (e.g., of blood) between the trunk and branch vessels of the bifurcated vessel. In some embodiments, a a curved portion of the periphery of the graft end faces the direction of flow of blood through the blood vessel.

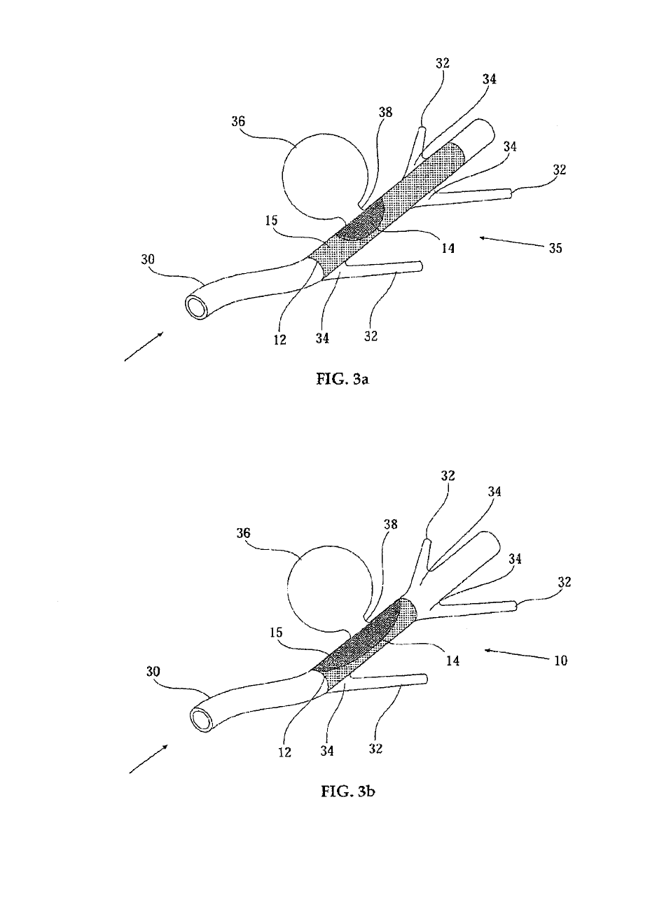

FIG. 3A depicts a bifurcated blood vessel with a trunk vessel 30, a plurality of branch vessels 32 and a plurality of bifurcation points 34. An aneurysm 36 is located on trunk vessel 30. An implantable graft-assembly 35 having a circular graft 14 is shown in an expanded state within trunk vessel 30. Implantable graft-assembly 35 is positioned within trunk vessel 30 such that graft 14 is positioned over neck 38 of aneurysm 36, and uncovered portion 15 of frame 12 is positioned over branch point 34, such that blood is allowed to flow through the struts of frame 12 into branch vessels 32.

FIG. 3B depicts a bifurcated blood vessel with a trunk vessel 30, a plurality of branch vessels 32 and a plurality of bifurcation points 34. An aneurysm 36 is located on trunk vessel 30. Implantable graft-assembly 10 such as depicted in FIG. 1A having a graft 14 with an elliptical periphery is shown in an expanded state within trunk vessel 30. Implantable graft-assembly 10 is positioned within trunk vessel 30 such that graft 14 is positioned over neck 38 of aneurysm 36, and uncovered portion 15 is positioned over branch point 34, such that blood is able to flow through the struts of frame 12 into branch vessels 32.

Deployment of an implantable graft-assembly having a graft that covers only a portion of the circumference of the respective frame (such as depicted in FIGS. 1A, 1B and 1C) requires that the graft be oriented properly over the neck of the aneurysm. To this end, it is required that a delivery system used for deploying such a graft-assembly be configured to control the radial orientation of the graft within the blood vessel. Generally, a device for deploying a graft-assembly in a vessel of a mammalian body in accordance with the teachings of the present invention, comprises a) an elongated delivery catheter with a distal end and a proximal end, including: i. a catheter-guiding guide wire lumen; and ii. a graft-assembly deploying mechanism; and b) a graft-assembly, including: iii. a radially expandable substantially tubular frame having a distal frame end and a proximal frame end; and iv. a graft associated with the frame, wherein a first portion of the surface area of the frame is covered by the graft and a second portion of the surface area of the frame is free of the graft the first portion having a circumferential section which is less than the entire circumference of the frame; the graft-assembly in an unexpanded state encircling the delivery catheter near the distal end of the delivery catheter and functionally associated with the graft-assembly deploying mechanism and the elongated catheter configured to control the radial orientation of the graft-assembly inside the body of a mammal when the graft-assembly is functionally associated with the graft-assembly deploying mechanism. In some embodiments, where the graft have an at least partially curved periphery, the graft-stent is oriented so that a curved part of the periphery is directed towards the distal end of the catheter. In some embodiments, where the graft have an at least partially curved periphery, the graft-stent is oriented so that a curved part of the periphery is directed towards the proximal end of the catheter.

In some embodiments, a delivery system used is configured to control the radial orientation of the graft by rotation of the implantable graft-assembly when inside the body during the deployment process. Generally, such deployment follows conventional procedures. For example, a catheter-guiding guide wire is backloaded into a delivery catheter having the implantable graft-assembly loaded over an inflatable balloon or on a self-expanding stent delivery system. The delivery catheter and the guide-wire are percutaneously introduced by means of a conventional Seldinger technique and a 6 to 10 French guiding catheter into the patient's arterial system. The guide-wire is advanced through the vasculature under fluoroscopic imaging until it crosses the target region, specifically across the neck of the aneurysm. The delivery catheter is advanced over the guide wire until the implantable graft-assembly is maneuvered into position at the desired location within the target region. The delivery system is used to rotate the graft-assembly to the desired position, across the neck of an aneurysm. The graft-assembly deploying mechanism is activated (e.g., a balloon is inflated or a securing mechanism of a self-expanding stent is released) to expand the frame of the graft-assembly, thereby pressing the graft against walls of the blood vessel and over the neck of the aneurysm so as to substantially seal or block, at least partially, the neck of the aneurysm. In some embodiments, rotating is with reference to an observable marker (functionally associated with, for example, the graft, the frame, the delivery system), for example a marker observable by a medical imaging modality such as staples 33 of graft-assembly 26 depicted in FIG. 1C.

If applicable the balloon or analogous components is deflated, and the delivery catheter and guide wire are removed, leaving the expanded implantable graft-assembly deployed in place, for example as depicted in FIGS. 3A and 3B.

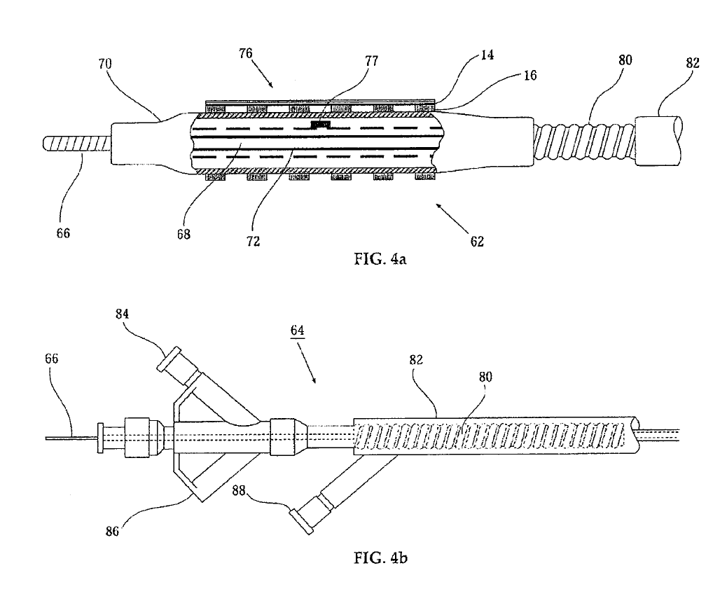

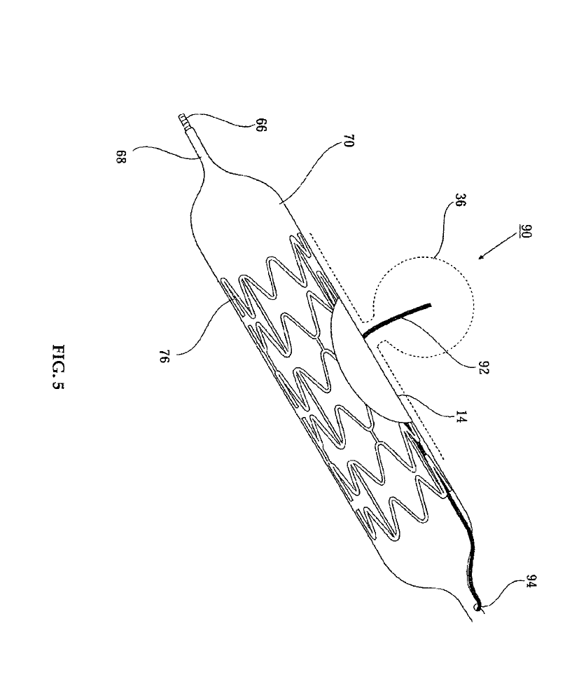

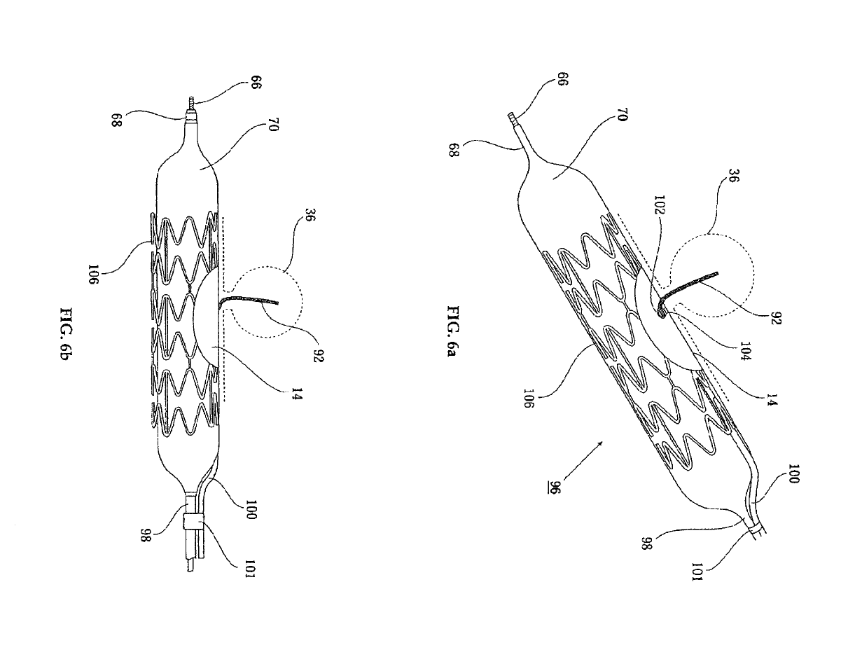

An embodiment of a delivery device configured to control the radial orientation of the graft by rotation of the implantable graft-assembly when inside the body during the deployment process includes a delivery catheter such as depicted in FIG. 4A (distal end 62 of the delivery catheter) and FIG. 4B (proximal end 64 of the delivery catheter).

Distal end 62 of the delivery catheter is similar to that of prior art balloon catheters known in the art of stent delivery, and includes the distal end of a guide wire 66 running through a guide wire lumen 68, around which is arranged a stent-expanding balloon 70 in fluid communication with an inflation/deflation lumen 72. Graft-assembly 76 (substantially similar to a graft-assembly depicted in FIG. 1A or 1C) is crimped over balloon 70 so that the center of the graft (not depicted) of graft-assembly 76 is over radio-opaque (and/or ultrasound opaque) marker 77. Proximal to balloon 70 is drive shaft 80 surrounded by external sleeve 82. Drive shaft 80 and external sleeve 82 are similar to corresponding components in the commercially available X-Sizer® Catheter System (ev3 corporation, Plymouth, Minn., USA) and allow rotation of drive shaft 80 inside sleeve 82 and consequently rotation of balloon 70 and graft-assembly 76.

Proximal end 64 of the delivery catheter is similar to that of prior art balloon catheters, and includes the proximal end of guide wire 66 entering guide wire lumen 68. Opposing balloon inlation/deflation port 84 is rotation handle 86. In fluid communication with the lumen of sleeve 82 is external sleeve infusion port 88.

Deployment of graft-assembly 76 using the delivery catheter depicted in FIGS. 4A and 4B is performed substantially as described above. Continuously, or only when it is desired to rotate drive shaft 80 or to purge air from the lumen of sleeve 82, a fluid such as heparinized saline is injected into port 88 and shaft 80 rotated with the help of port 84 and rotation handle 86. The degree of rotation and accurate positioning of the graft is performed with reference to marker 77 observed with the help of an appropriate medical imaging modality.

In some embodiments, a drive shaft is provided with a ferromagnetic portion along a carotid section of the drive shaft, that is a portion of the drive shaft that is located in the carotid artery during deployment of a graft-assembly and a powerful adjustable magnet placed around the neck of the subject being treated. When the graft-assembly is across the neck of the aneurysm, the adjustable magnet is used to apply a force (torque) to the ferromagnetic portion of the drive shaft, causing the drive shaft and consequently the graft to rotate. The adjustable magnet may be configured to ensure accurate positioning of the graft across the neck of the aneurysm.

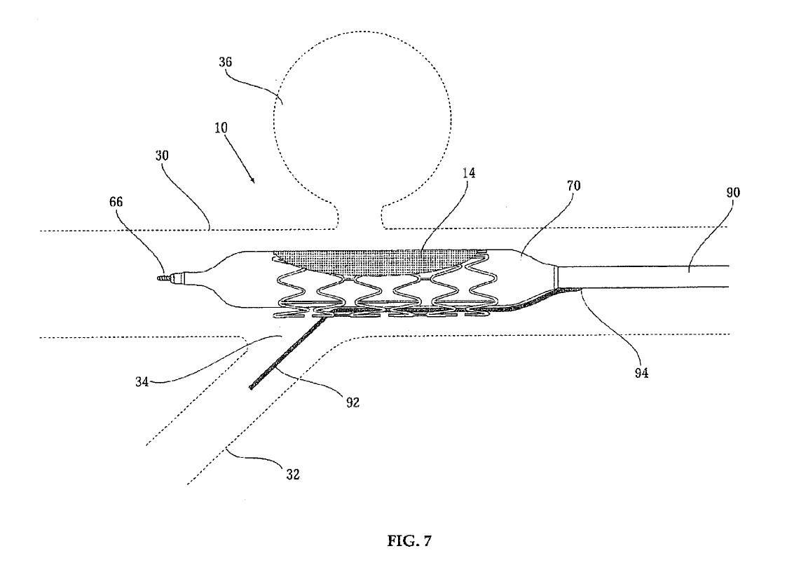

In some embodiments, a delivery system is configured to control radial orientation of the graft with reference to an orientation guide wire 92 as depicted in FIG. 5, FIGS. 6A and 6B or FIG. 7, for example as described in PCT patent application IL2007/000140 of the Inventor. In some such embodiments, the delivery system comprises a catheter guiding guide wire 66, an orientation guide wire 92 and an appropriately modified delivery catheter. In some such embodiments, the delivery catheter includes a region near a distal end of the delivery catheter on which the implantable graft-assembly is positionable for deployment (for example over a balloon for inflating the expandable frame of a graft-assembly, a first guide wire lumen for engaging the catheter guiding guide wire running from a proximal end of the delivery catheter through a distal end of the delivery catheter; a second orientation guide wire lumen for engaging the orientation guide wire, the orientation guide wire lumen including a proximal port near the proximal end of the delivery catheter and a distal port emerging near the distal end of the delivery catheter, proximal to the region on which the graft-assembly is positionable.

In some embodiments, the graft-assembly is mounted in an unexpanded state (e.g., crimped) onto the region so as to be functionally associated with the graft-assembly deploying mechanism so that the distal port is in-line with the graft, preferably in-line with the longitudinal axis of the graft.

The catheter guiding guide wire is placed in the blood vessel across the neck of the aneurysm and the orientation guide wire is placed in the blood vessel and into the aneurysm through the neck of the aneurysm. The delivery catheter with the graft-assembly is mounted onto the two guide wires: the catheter guiding guide wire in the first lumen and the orientation guide wire in the second lumen.

By guiding the delivery catheter along the the two guide wires, the graft is maneuvered to the proximity of the neck of the aneurysm along the catheter guide wire and the orientation guide wire, ensuring that the graft is aligned with the neck of the aneurysm. The catheter guiding guide wire passes through the entire delivery catheter from outside the patient all the way through the end of the delivery catheter, including through the region of the delivery catheter over which the graft-assembly is located, analogously to guide wires known in the art of stent delivery. In contrast, the orientation guide wire passes through the delivery catheter and emerges proximally to the region of the delivery catheter over which the implantable graft-assembly is located mounted on the same side where the graft is positioned and into the aneurysm through the neck of the aneurysm. As the delivery catheter progresses along the two guide wires, the entire delivery catheter is directed by the orientation guide wire in such a way that the graft is properly located with respect to the neck of the aneurysm.

When in place, the frame of the graft-assembly is expanded, thereby pressing the graft against walls of the blood vessel and across the neck of the aneurysm so as to substantially seal the neck of the aneurysm.

A first embodiment of a delivery system including two guide wires where the orientation guide wire passes over the outside of a graft of a graft-assembly is depicted in FIG. 5. In FIG. 5 is depicted the distal end of delivery catheter 90. Delivery catheter 90 is similar to that of prior art balloon catheters known in the art of stent delivery, and includes the distal end of a catheter guiding guide wire 66 running through a guide wire lumen 68 from the proximal end (not depicted) of guide wire lumen 68 out through the distal end of guide wire lumen 68 at the distal end of catheter 90. Graft-assembly 76 including a substantially circular graft 14 is crimped over a balloon 70, balloon 70 configured to function in the usual way. Unlike prior art stent-delivery catheters, delivery catheter 90 includes an additional distal orientation guide wire lumen that runs from the proximal end of delivery catheter 90 (not depicted) to an orientation guide wire port 94 that is positioned proximally to balloon 70.

When delivery catheter 90 is mounted onto orientation guide wire 92, orientation guide wire is passed through gaps in the frame of graft-assembly 76 just proximal to the proximal edge of graft 14 so as to pass underneath the frame of graft-assembly 76, and then orientation guide wire is threaded into distal orientation guide wire port 90. Thus and as depicted in FIG. 5, an orientation guide wire 92 passes through the orientation guide wire lumen of delivery catheter 90, emerges from distal orientation guide wire port 94, passes underneath the frame of graft-assembly, passes through gaps in the frame to pass over graft 14 and into aneurysm 36.

Deployment of graft-assembly 76 using delivery catheter 90 depicted in FIG. 5 is performed substantially as described above. As the distal end of orientation guide wire 92 is located inside aneurysm 36, orientation guide wire 92 forces distal guide wire port 94 and consequently also graft 14 to be oriented properly vis a vis aneurysm 36. Generally, orientation guide wire 92 is withdrawn from aneurysm 36 and away from balloon 70 prior to expanding of the frame of graft-assembly 72 so as not to interfere with the expansion.

A second embodiment of a delivery system including two guide wires where an orientation guide wire 92 passes between a delivery catheter 96 and a graft-assembly 106 to emerge through an alignment hole 102 reinforced with radio opaque grommet 104 penetrating through a graft 14 having a curved periphery with an elliptical shape is depicted in FIGS. 6A and 6B. FIGS. 6A and 6B depict the distal end of delivery catheter 96. Delivery catheter 96 is similar to that of prior art balloon catheters known in the art of stent delivery, and includes the distal end of a catheter guiding guide wire 66 running through a guide wire lumen 68 inside a main catheter shaft 98 from the proximal end (not depicted) of guide wire lumen 68 out through the distal end of guide wire lumen 68 at the distal end of delivery catheter 96. Graft-assembly 106 including graft 14 is crimped over a balloon 70, balloon 70 configured to function in the usual way.

Unlike prior art stent-delivery catheters, delivery catheter 96 includes an additional orientation guide wire shaft 100 that is substantially a tube that defines an orientation guide wire lumen. Orientation guide wire shaft 100 is secured to main catheter shaft 98 at point 101 and then runs over balloon 70 to approximately the middle of balloon 70.

The distal end of orientation guide wire shaft 100 where the orientation guide wire lumen ends (not depicted) defines a distal orientation guide wire port that is hidden from view in FIGS. 6A and 6B underneath graft 14 of graft-assembly 106. In FIGS. 6A and 6B, graft-assembly 106 is crimped over balloon 70 and over orientation guide wire shaft 100 so that alignment hole 102 is substantially above the distal end of orientation guide wire shaft 100. In FIGS. 6A and 6B, an orientation guide wire 92 passes through the orientation guide wire lumen of orientation guide wire shaft 100 of delivery catheter 96 from the proximal end of orientation guide wire shaft (not depicted) to emerge from the distal orientation guide wire port through alignment hole 102 in graft 14 to pass into aneurysm 36.

Deployment of graft-assembly 106 using delivery catheter 96 depicted in FIGS. 6A and 6B is performed substantially as described above. As the distal end of orientation guide wire 92 is located inside aneurysm 36, orientation guide wire 92 forces the guide wire port of orientation guide wire shaft 100 and consequently also graft 14 to be oriented properly vis a vis aneurysm 36. Orientation guide wire 92 is withdrawn from aneurysm 36 either prior or subsequently to expanding of the tubular frame of graft-assembly 106.

In some embodiments, a delivery device is configured for radially orientating a graft-assembly by including an orientation guide wire lumen including a proximal port and a distal port 94 near the distal end of the delivery catheter, where the distal port 94 of the orientation guide wire lumen is in-line with the second portion of the surface area of the frame, that is to say the portion of the frame that is free of the graft. Such an embodiment is schematically depicted in FIG. 7 where delivery catheter 90 is used to deploy graft 14 of graft-assembly 10 across the mouth of aneurysm 36, where aneurysm is close to a bifurcation 34, on the luminal wall of trunk vessel 30 oriented at about 180° from branch vessel 32.

Similarly to the described above, a catheter-guiding guide wire 66 is directed in the usual way through trunk vessel 30 past aneurysm 36 and bifurcation point 34. Orientation guide wire 92 is directed in the usual way into branch vessel 32.

Delivery catheter 90 is loaded onto catheter-guiding guide wire 66 by threading the distal end of catheter-guiding guide wire 66 into the catheter guiding guide wire lumen of delivery catheter 90.

Delivery catheter 90 is loaded onto orientation guide wire 92 by passing orientation guide wire 92 through the gaps in the frame of graft-assembly 10 in line with distal port 94 of orientation guide wire lumen, passing the orientation guide wire between the frame and balloon 70 and then threading the orientation guide wire 92 into the orientation guide wire lumen of delivery catheter 90.

Deployment of graft-assembly 10 using delivery catheter 90 as depicted in FIG. 7 is performed substantially as described above. Delivery catheter 90 is advanced along guide wires 66 and 92. As the distal end of orientation guide wire 92 is located inside branch vessel 32, orientation guide wire 92 forces distal port 94 of orientation guide wire lumen to be oriented on the side of branch vessel 32, and therefore graft 14 to be oriented properly vis a vis aneurysm 36. Once graft 14 is properly positioned, orientation guide wire 92 is withdrawn from branch vessel 92 either prior or subsequently to expanding of the tubular frame of graft-assembly 10.

As is clear to one skilled in the art upon perusal of the above, a graft is oriented at a section of a blood vessel that is determined by the angle at which a distal port of the orientation guide wire lumen is from the longitudinal axis of the graft. Thus, it is advantageous to provide a number of similar devices comprising a graft-assembly mounted on a delivery catheter, the devices differing by the angle at which the distal port of the orientation guide wire lumen is oriented from the longitudinal axis of the graft. For example, in one device the angle is about 90°, in a second device the angle is about 180° and in a third device the angle is about 270° (90° in the opposite direction) from the longitudinal axis of the graft. Medical personnel treating a subject using the teachings of the present invention are then able to select a suitable branch into which to direct the orientation guide wire, and then to select the appropriate device which will allow the most suitable orientation of the graft during deployment of the graft-assembly to most effectively treat the subject.

Deployment of balloon-expandable graft-assemblies in accordance with some embodiments of the teachings of the present invention is described above with reference to FIGS. 4, 5, 6 and 7. As noted above, some embodiments of the present invention relate to self-expanding graft-assemblies and to devices for deplying such graft-assemblies.

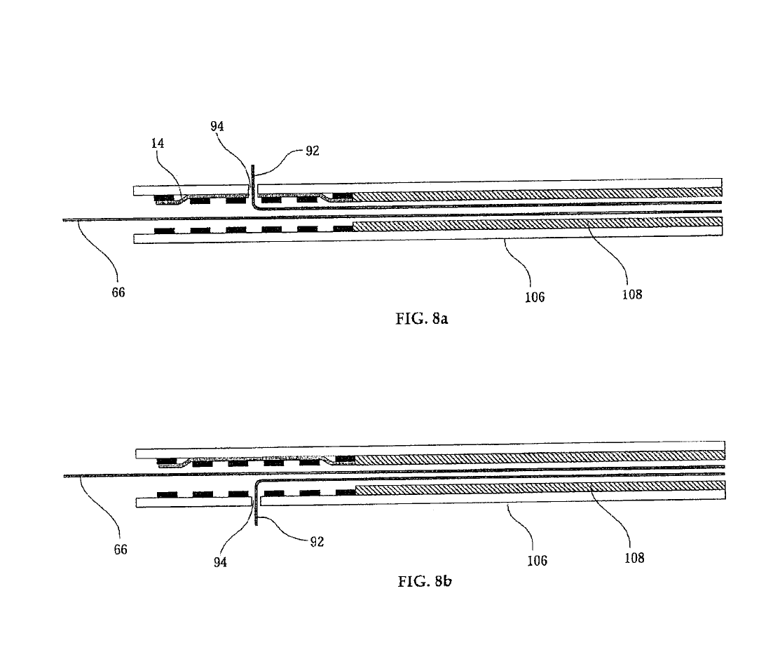

In FIG. 8A is depicted a device for deploying a self-expanding graft-assembly that is analogous to the described with reference to FIGS. 6A and 6B for a balloon expandable graft assembly. The device of FIG. 8A comprises an elongated delivery catheter including a delivery sheath 106 with a slidingly associated coaxial push tube 108 as a component of a graft-assembly deploying mechanism. The bore of push tube 108 defines a catheter-guiding guide wire lumen and an orientation guide wire lumen. At the distal end of the delivery catheter is held a self-expanding graft-assembly comprising six expandable rings associated with a graft 14 part of which contacts the outside surface of the stent and part of which contacts the inside surface of the stent, similar to the depicted in FIG. 1G. Graft 14 is provided with an alignment hole (similar to 102 depicted in FIG. 6A) that is aligned with a distal orientation guide wire port 94 that passes through the wall of delivery sheath 106.

For deployment of the graft-assembly, a catheter guiding guide wire 66 is passed through the lumen of delivery sheath 106 and push tube 108, in the usual way, while an orientation guide wire 92 (which distal tip is located inside an aneurysm) is passed through distal orientation guide wire port 94 in delivery sheath 106, the alignment hole in graft 14 and then through the lumen of delivery sheath 106 and push tube 108. The catheter is advanced along guide wires 66 and 92 substantially as described above with reference to FIGS. 6A and 6B, ensuring that graft 14 faces the neck of the aneurysm. Orientation guide wire 92 is withdrawn and then push tube 108 used to push the graft-assembly out of delivery sheath 106 so that graft 14 blocks the neck of the aneurysm.

In FIG. 8B is depicted a device for deploying a self-expanding graft-assembly that is analagous to the describe with reference to FIG. 7 for a balloon-expandable graft-assembly. The device of FIG. 8B comprises an elongated delivery catheter including a delivery sheath 106 with a slidingly associated coaxial push tube 108 as a component of a graft-assembly-deploying mechanism. The bore of push tube 108 defines a catheter-guiding guide wire lumen and an orientation guide wire lumen. At the distal end of the delivery catheter is held a self-expanding graft-assembly comprising six expandable rings associated with a graft 14 part of which contacts the outside surface of the stent and part of which contacts the inside surface of the stent, similar to the depicted in FIG. 1G. Located at about 180° from the longitudinal axis of graft 14 is a distal orientation guide wire port 94 that passes through the wall of delivery sheath 106.

For deployement of the graft-assembly, a catheter guiding guide wire 66 is passed through the lumen of delivery sheath 106 and push tube 108, in the usual way, while an orientation guide wire 92 (which distal tip is located inside a vessel branching opposite the neck of an aneurysm) is passed through distal orientation guide wire port 94 in delivery sheath 106, through a portion of the surface area of the expandable frame of the graft-assembly (the stent) that is free of graft 14 and then through the lumen of delivery sheath 106 and push tube 108. The catheter is advanced along guide wires 66 and 92 as described above with reference to FIG. 7, ensuring that graft 14 faces the neck of the aneurysm. Push tube 108 is used to push the graft-assembly out of delivery sheath 106 so that graft 14 blocks the neck of the aneurysm.

Analogous to the described with reference to a balloon-expandable graft-assembly 10 in FIG. 7, it is advantageous to provide a number of devices such as discussed with reference to FIG. 8b comprising a self-expanding graft-assembly mounted on a delivery catheter, the devices differing by the angle at which the distal port of the orientation guide wire lumen is oriented from the longitudinal axis of the graft. For example, in one device the angle is about 90°, in a second device the angle is about 180° (as depicted in FIG. 8B) and in a third device the angle is about 270° (90° in the opposite direction) from the longitudinal axis of the graft.

The deployment of graft-assemblies described above was of various graft assemblies including grafts with an at least partially curved periphery. It is clear to one skilled in the art that the methods of deployment and devices therefore may be modified to deploy graft-assemblies including grafts not having curved peripheries, for example, graft assemblies described in WO 2007/088549 of the Inventor.

The invention was described above with reference to the vascular system and blood vessels. However, the present invention with suitable modification, is also suitable for implementation in other bodily vessels.

Exemplary embodiments of the invention are discussed herein with reference to specific materials, methods and examples. The material, methods and examples discussed herein are illustrative and not intended to be limiting. In some embodiments, methods and materials similar or equivalent to those described herein are used in the practice or testing of embodiments of the invention. It is to be understood that the invention is not necessarily limited in its application to the details of construction and the arrangement of the components and/or methods set forth in the following description and/or illustrated in the drawings. The invention is capable of other embodiments or of being practiced or carried out in various ways.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the present invention.

To the extent that section headings are used, they should not be construed as necessarily limiting.

1-25. (canceled)

26. An implantable graft-assembly comprising:
a) a radially expandable substantially tubular frame having a distal frame end and a proximal frame end; and
b) a graft associated with said frame having an at least partially curved periphery, wherein a first portion of the surface area of said frame is covered by said graft and a second portion of the surface area of said frame is free of said graft said first portion having a circumferential section which is less than the entire circumference of said frame.

27. The graft-assembly of claim 26, wherein said periphery of said graft is substantially entirely curved.

28. The graft-assembly of claim 26, wherein said graft comprises a sheet of material having a shape selected from the group consisting of circles, ovals, ellipses, oblate ovals, oblate ellipses and oblate circles.

29. The graft-assembly of claim 26, wherein a sector spanning at least about 90° of said periphery of said graft is curved.

30. The graft-assembly of claim 26, said first portion covering a circumferential section comprising not more than about 330° of the entire circumference of said frame.

31. The graft-assembly of claim 26, said graft associated with said frame so that a said curved portion of said periphery of said graft is directed towards an upstream end of said frame.

32. The graft-assembly of claim 26, further comprising an alignment hole penetrating through said graft.

33. The graft-assembly of claim 26, wherein said graft contacts an inner surface of said frame.

34. The graft-assembly of claim 26, wherein said graft contacts an outer surface of said frame.

35. The graft-assembly of claim 26, wherein said graft contacts both an inner surface and an outer surface of said frame.

36. A method of making an implantable graft-assembly; comprising:
a) providing a radially expandable substantially tubular frame;
b) providing a graft comprising a sheet of material suitable for use as an implantable graft having an at least partially curved periphery; and
c) associating said graft with a surface of said frame so that said graft covers a first portion of the surface area of said frame and a second portion of the surface area of the frame is free of the graft, wherein said first portion has a circumferential section which is less than the entire circumference of said frame.

37. The method of claim 36, wherein said periphery of said graft is substantially entirely curved.

38. The method of claim 36, wherein said graft comprises a sheet of material having a shape selected from the group consisting of circles, ovals, ellipses, oblate ovals, oblate ellipses and oblate circles.

39. The method of claim 36, wherein a sector spanning at least about 90° of said periphery of said graft is curved.

40. The method of claim 36, said first portion covering a circumferential section comprising not more than about 330° of the entire circumference of said frame.

41. The method of claim 36, said graft associated with said frame so that a said curved portion of said periphery of said graft is directed towards an upstream end of said frame.

42. The method of claim 36, further comprising an alignment hole penetrating through said graft.

43. The method of claim 36, wherein said graft contacts an inner surface of said frame.

44. The method of claim 36, wherein said graft contacts an outer surface of said frame.

45. The method of claim 36, wherein said graft contacts both an inner surface and an outer surface of said frame.

46. A method of treating an aneurysm, comprising:
a) providing an implantable graft-assembly comprising:
i) a radially expandable substantially tubular frame having a distal frame end and a proximal frame end; and
ii) a graft having an at least partially curved periphery associated with said frame, wherein a first portion of the surface area of said frame is covered by said graft and a second portion of the surface area of said frame is free of said graft, said first portion having a circumferential section which is less than the entire circumference of said frame;
b) providing a delivery system for deploying said implantable graft-assembly within a blood vessel on which an aneurysm is located; and
c) deploying said implantable graft-assembly within said blood vessel using said delivery system, such that said first portion of said frame covered by said graft is positioned across a neck of said aneurysm.

47. A device for deploying a graft-assembly in a vessel of a mammalian body, comprising:
a) an elongated delivery catheter with a distal end and a proximal end, including:
i. a catheter-guiding guide wire lumen; and
ii. a graft-assembly deploying mechanism; and
b) a graft-assembly, including:
iii. a radially expandable substantially tubular frame having a distal frame end and a proximal frame end; and
iv. a graft associated with said frame, wherein a first portion of the surface area of said frame is covered by said graft and a second portion of the surface area of said frame is free of said graft said first portion having a circumferential section which is less than the entire circumference of said frame
said graft-assembly in an unexpanded state encircling said delivery catheter near said distal end of said delivery catheter and functionally associated with said graft-assembly deploying mechanism; and
said elongated catheter configured to control the radial orientation of said graft-assembly inside the body of a mammal when said graft-assembly is functionally associated with said graft-assembly deploying mechanism
said graft having an at least partially curved periphery.

48. The device of claim 47, said graft-stent oriented so that a curved part of said periphery is directed towards said distal end of said catheter.

49. The device of claim 47, said graft-stent oriented so that a curved part of said periphery is directed towards said proximal end of said catheter.

50. The device of claim 47, said configuration for radially orienting said graft-assembly comprises a rotation mechanism configured for controllable rotation of said graft-assembly inside a body.

51. The device of claim 47, said configuration for radially orienting said graft-assembly comprising an orientation guide wire lumen associated with said delivery catheter, including a proximal port and a distal port near said distal end of said delivery catheter, said distal port in-line with said graft.

52. The device of claim 51, said graft comprising an alignment hole penetrating through said graft and said distal port in-line with said alignment hole.

53. The device of claim 51, said configuration for radially orienting said graft-assembly comprising an orientation guide wire lumen associated with said delivery catheter, including a proximal port and a distal port near said distal end of said delivery catheter, said distal port in-line with said second portion of the surface area of said frame that is free of said graft.

54. A device for deploying a graft-assembly in a vessel of a mammalian body, comprising:
a) an elongated delivery catheter with a distal end and a proximal end, including:
i. a catheter-guiding guide wire lumen; and
ii. a graft-assembly deploying mechanism; and
b) a graft-assembly, including:
iii. a radially expandable substantially tubular frame having a distal frame end and a proximal frame end; and
iv. a graft associated with said frame, wherein a first portion of the surface area of said frame is covered by said graft and a second portion of the surface area of said frame is free of said graft said first portion having a circumferential section which is less than the entire circumference of said frame
said graft-assembly in an unexpanded state encircling said delivery catheter near said distal end of said delivery catheter and functionally associated with said graft-assembly deploying mechanism; and
said elongated catheter configured to control the radial orientation of said graft-assembly inside the body of a mammal when said graft-assembly is functionally associated with said graft-assembly deploying mechanism, said configuration for radially orienting said graft-assembly comprising an orientation guide wire lumen associated with said delivery catheter, including a proximal port and a distal port near said distal end of said delivery catheter.

55. The device of claim 54, said graft having an at least partially curved periphery.

56. The device of claim 55, said graft-stent oriented so that a curved part of said periphery is directed towards said distal end of said catheter.

57. The device of claim 55, said graft-stent oriented so that a curved part of said periphery is directed towards said proximal end of said catheter.

58. The device of any of claim 54, said distal port in-line with said graft.

59. The device of claim 58, said graft comprising an alignment hole penetrating through said graft and said distal port in-line with said alignment hole.

60. The device of claim 58, said distal port in-line with a longitudinal axis of said graft.

61. The device of claim 54, said distal port in-line with said second portion of the surface area of said frame that is free of said graft.

62. The device of claim 61, said distal port at about 90° from a longitudinal axis of said graft.

63. The device of claim 61, said distal port at about 180° from a longitudinal axis of said graft.
